(19) United States
(12) Patent Application Publication
(10) Pub. No.: US 2011/0144347 A1
(43) Pub. Date: Jun. 16, 2011

(54) PROCESS FOR THE EPIMERIZATION OF ATOVAQUONE ISOMER, ATOVAQUONE INTERMEDIATES AND MIXTURE THEREOF

(75) Inventors: Fuqiang Zhu, Shanghai (CN); Michel Bekhazi, Pointe Claire (CA)

(73) Assignee: CHEMAGIS LTD., Bnei Brak (IL)

(21) Appl. No.: 13/001,156

(22) PCT Filed: Jun. 30, 2008
(86) PCT No.: PCT/IL2008/000892

§ 371 Date: Feb. 24, 2011

(51) Int. Cl. C07D 213/70; C07C 46/10

(52) U.S. Cl. 546/302; 552/297; 552/298

(57) ABSTRACT

Provided is a process for the epimerization of the cis isomer of atovaquone, atovaquone intermediates and isomeric mixtures thereof into their corresponding trans-isomers resulting in higher yield of pure atovaquone.

### FIELD OF THE INVENTION

The field of the invention relates to the epimerization process of atovaquone isomer, atovaquone intermediates and isomeric mixtures thereof.

### BACKGROUND OF THE INVENTION

Atovaquone, trans-(2-[4-(4-Chlorophenyl)cyclohexyl]-3-hydroxy-1,4-naphthoquinone (compound I), is represented by the following structural formula:

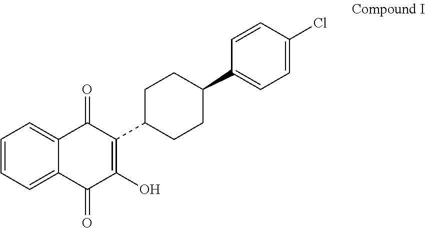

Atovaquone is the active ingredients in two drugs which are marketed in the United States, Europe and other countries by GSK. The first drug is an oral suspension (750 mg/5 mL) under the trade name Mepron® and is indicated for the treatment and prophylaxis of Pneumocystis carinii infection. The second drug is a combination with proguanil hydrochloride, under the brand name Malarone® for the prophyaxis of Malaria. Malaron® is supplied as an oral tablet containing 250 mg of atovaquone and 100 mg of proguanil hydrochloride and a pediatric dosage containing 62.5 mg of atovaquone and 25 mg of proguanil hydrochloride.

The synthesis of atovaquone was disclosed in U.S. Pat. No. 4,981,874, herein referred to as the '874 patent. The process is illustrated in scheme 1,

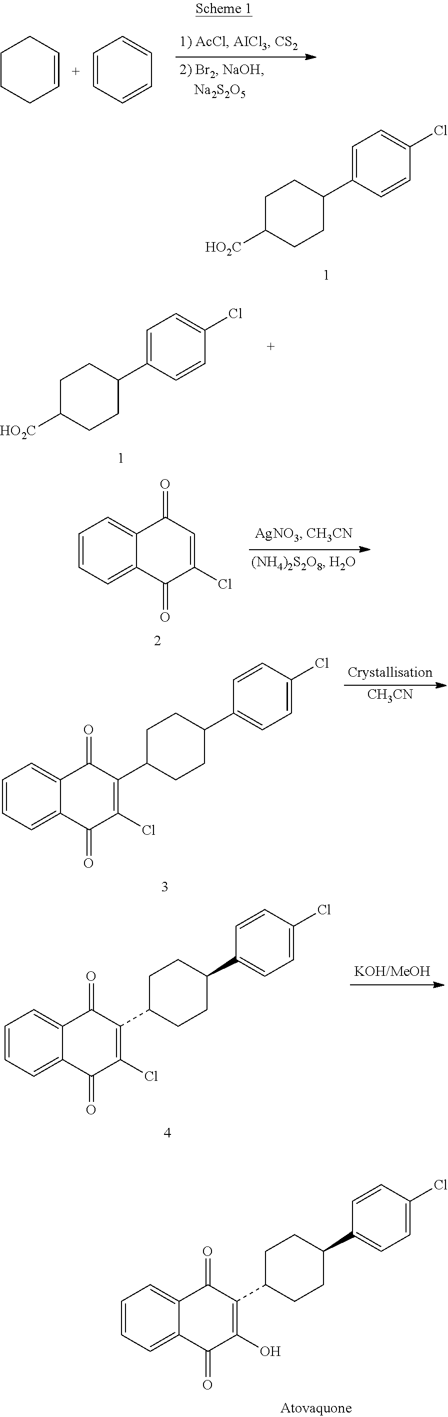

The process described in the 874' patent is reported to give a low yield of atovaquone (4% total yield of atovaquone calculated from the last two steps).

An additional process is disclosed in Tetrahedron Letters 39 (1998) 7629-7632 (David R. Williams and Michael P. Clark). The mixture of cis and trans isomers of formula 3 are produced by reacting the oxalate of formula 5, with 2-chloro-1,4-naphthquinone, a compound of formula 2, in the presence of silver nitrate, ammonium per sulphate and a phase transfer catalyst such as Adogen 464. The crude produced is purified by flash chromatography using ethyl acetate/hexanes to isolate 2-[4-(4-chlorophenyl)cyclohexyl-3-chloro-1,4-naphtoquinone, compound of formula 3 (1.3 to 1 ratio of trans/cis-isomers, 43% yield) and the ester by-product, 3-chloro-1,4-dihydro-1,4-dioxo-4-(4-chlorophenyl)cyclohexyl ester-2-naphthalenecarboxylic acid of formula 6 (38% yield). Finally the conversion to atovaqoune was performed as described in the '874 patent mentioned above. The process is illustrated in scheme 2.

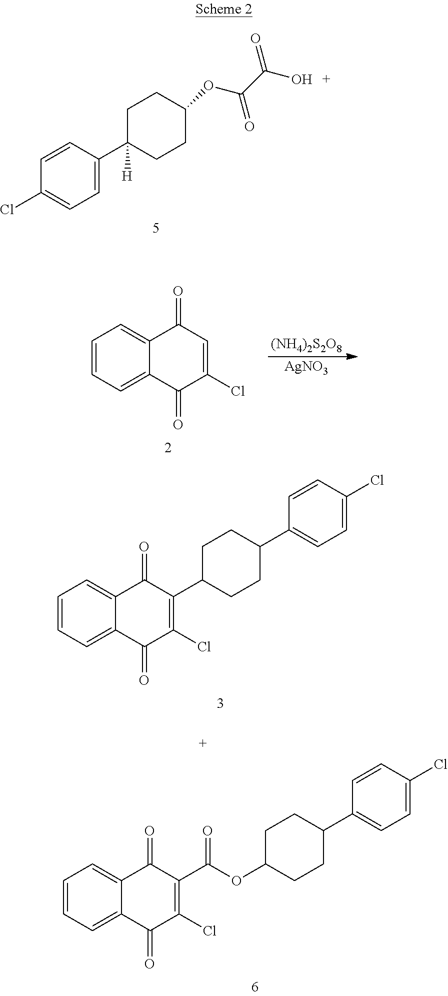

The disadvantage of the above process is that the resulting product 3 is purified by column chromatography, which is time and solvents consuming and difficult to apply in industrial large scale production. Further more compound 3, which is used in the next step of the conversion to atovaquone, is a mixture of the cis and trans isomers and is expected to provide low yield of atovaquone as described in the 874' patent.

Epimerization of cis-2-(4-t-butylcyclohexyl)-3-hydroxy-1,4-naphthquinone, compound of formula 7, which is structurally related to atovaquone, is documented in European Patent No. 77551 (Alan T. Hudson and Anthony W. Randall), having a priority date of Oct. 16, 1981 (refer herein as the 551' patent). The reaction is carried out in sulfuric acid at a temperature range of 50 to 70° C., from 4 hours to 4 days. The isomer ratios ranges between 92:8 to 99.4:0.6 (trans:cis) depending on the temperature and reaction time. Higher purities can be obtained by recrystallization and/or column chromatography. The yield of the epimerization is not reported, nor the details for the purification process. The inventors of the 551' patent published a later detailed publication (Alan T. Hudson and Anthony W. Randall Eur. J. Med. Chem 1986, p 271-275), in which they contradict the disclosure of said patent. They reported there that an attempt for the epimerization of cis-2-hydroxy-3-(4-t-butylcyclohexyl)-1,4-naphthquinone, compound of formula 7, in sulfuric acid at 55° C. for 40 hours failed to give the desired trans-2-hydroxy-3-(4-t-butylcyclohexyl)-1,4-naphthquinone. It has been also reported that treatment of the individual isomers in sulfuric acid under those conditions showed that wherein the trans form can be recovered unchanged, the cis form (example b, page 275) is converted to the products of the intramolecular cyclization, compounds of formula 8, 9 and 2-hydroxy-1,4-naphthoquinone of formula 10 (scheme 3). Furthermore, the product analysis of the mixture of cis and trans-2-hydroxy-3-(4-t-butylcyclohexyl)-1,4-naphthquinone (52:48 ratio of cis/trans) after reaction with sulfuric acid revealed the trans product (40.5% yield), compound 8 (35.5% yield), compound 9 (1.1% yield) and 2-hydroxy-1,4-naphthoquinone of formula 10 (2.7% yield), but no cis isomer was detected.

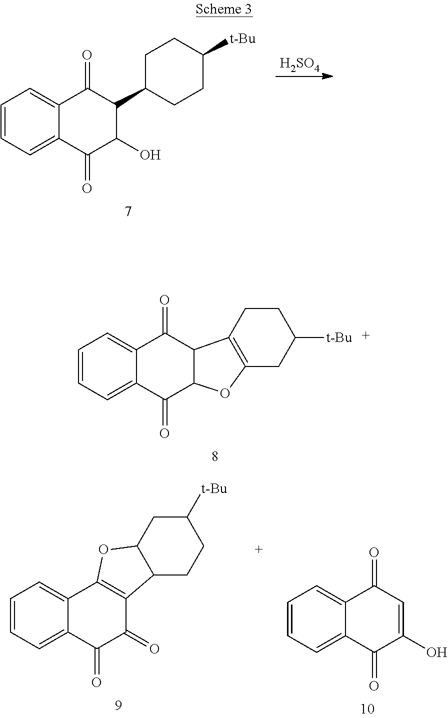

There are further problems associated with the production of atovaquone, particularly for the separation of isomers, which tend to produce poor yields. The processes described above are reported to give low yields of Atovaquone while the epimerization process is reported to be unsuccessful. Therefore, there is an unmet need for an improved process which provides higher yields of pure atovaquone.

### SUMMARY OF THE INVENTION

The present invention provides an improved process for the epimerization of cis-1,4-naphthoquinones of formula (II), into trans-1,4-naphthoquinones of formula (I), wherein X is selected from among a hydroxyl, a halogen or SY, and Y is substituted or unsubstituted, aryl and heterocycloalkyl.

comprising:
a) reacting the compound of formula (II) with a strong acid;
b) quenching the reaction mixture; and
c) isolating the compound of formula (I)

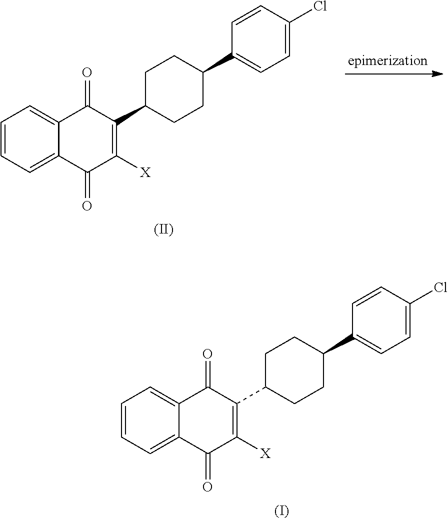

The present invention further provides a process for the epimerization of a mixture of cis and trans compounds of formula (III) into the trans compounds of formula (I) comprising:
a) reacting the compound of formula (III) with a strong acid,
b) quenching the reaction mixture; and
c) isolating the compound of formula (I).

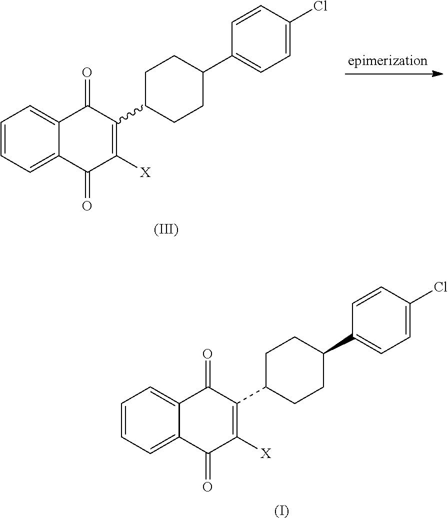

### BRIEF DESCRIPTION OF THE DRAWINGS

FIG. 1: depicts the HPLC chromatogram of the cis-(2-[4-(4-Chlorophenyl)cyclohexyl]-3-hydroxy-1,4-naphthoquinone used as starting material in example 1.

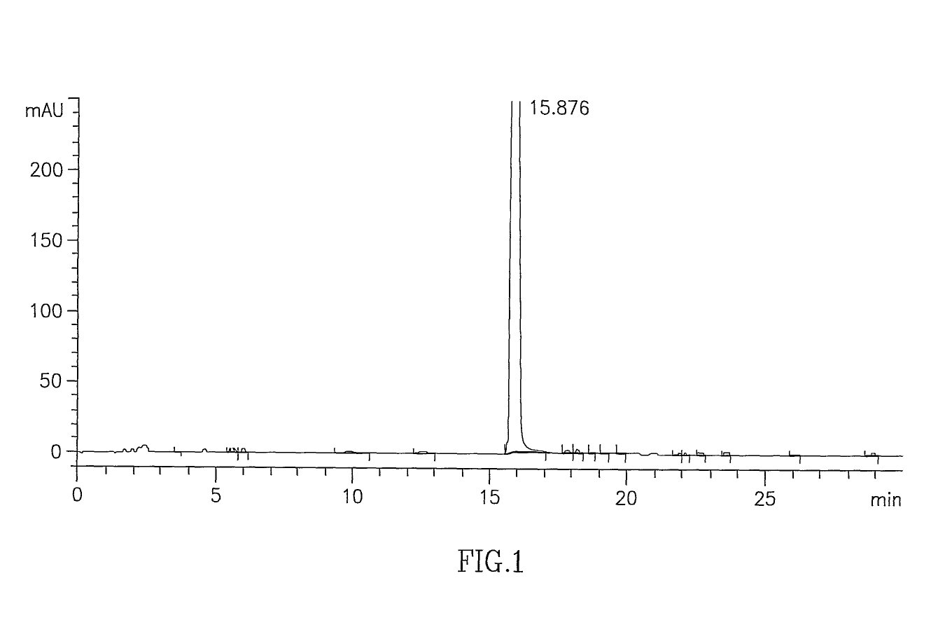

FIG. 2: depicts the HPLC chromatogram of the trans-(2-[4-(4-Chlorophenyl)cyclohexyl]-3-hydroxy-1,4-naphthoquinone product obtained by example 1.

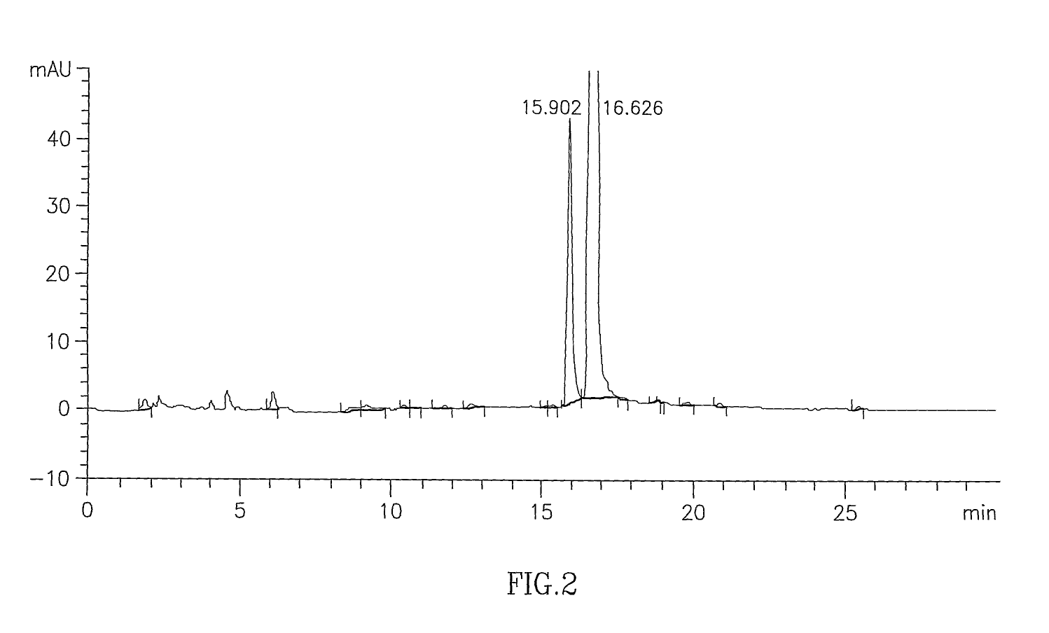

### DETAILED DESCRIPTION OF THE INVENTION

It has surprisingly been found that reacting the cis isomer of atovaquone, atovaquone intermediates or isomeric mixtures thereof with a strong acid resulted in a clean epimerization to the corresponding trans isomer and thus to high yields of the atovaquone obtained in the process of the present invention.

The following are definitions of terms used in this specification. The initial definition provided for a group or term herein applies to that group or term throughout the present specification, individually or as part of another group, unless otherwise indicated.

The term “epimerization”, as used herein, refers to a process in which only one of the chiral centers in an optically active compound changes its configuration.

The term “strong acid”, as used herein, refers to any acid with a pKa of less than 3.

The term “aryl” refers to aromatic cyclic groups which contain 6 to 14 carbon atoms.

The term “halogen” refers to fluorine, chlorine, bromine and iodine.

The term “heterocyclic” refers to fully saturated or unsaturated, including aromatic cyclic groups, which have at least one heteroatom in at least one carbon atom-containing ring. Each ring of the heterocyclic group containing a heteroatom may have 1, 2, 3 or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. The heterocyclic group may be attached at any heteroatom or carbon atom of the ring or ring system.

The invention provides a process for the epimerization of cis-1,4-naphthoquinones of formula (II), into trans-1,4-naphthoquinones of formula (I), wherein X is selected from among a hydroxyl, a halogen or SY, and Y is substituted or unsubstituted, aryl and heterocycloalkyl, comprising:
a) reacting the compound of formula (II) with a strong acid,
b) quenching the reaction mixture; and
c) isolating the compound of formula (I).

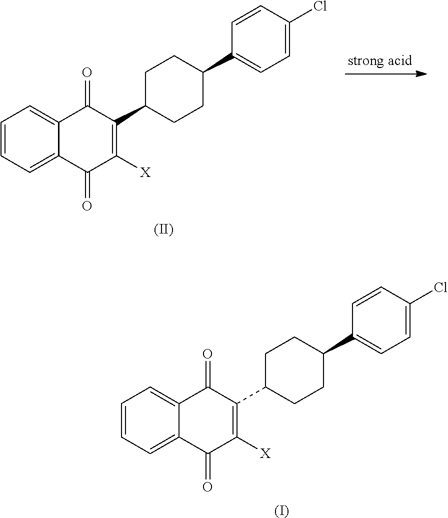

Suitable non limiting examples of strong acids are: sulfuric acid and methanesulphonic acid, preferably sulfuric acid.

The molar ratio of the strong acid (e.g sulfuric acid) to the 1,4-naphthoquinone of formula (II) can be from about 4:1 to about 40:1, preferably about 30:1.

The process may be carried out at a temperature range of 5-35° C., preferably at 15-18° C. and the reaction mixture is stirred for less than one hour.

In accordance with the present invention step (b) of quenching the reaction mixture, might be carried out by pouring the mixture into ice, and further stirring for 20 minutes. Step (b) may also be carried out by pouring the mixture into water at 0° C., and further stirring for 20 minutes.

In accordance with the present invention, after the reaction mixture is quenched, the isolation step (c) comprises the following steps:

(i) collecting the solid by flirtation, washing and drying to yield the 1,4-naphthoquinone of formula (I); and optionally

(ii) recrystallization of the obtained 1,4-naphthoquinone of formula (I).

The isolated 1,4-naphthoquinone of formula (I), obtained in step (c) (i) may be dried by using conventionally known methods. The drying procedure may be carried out by increasing the temperature or by reducing the pressure or a combination of both. Non limiting examples of drying technologies or equipment usable in context of the present invention include: rotary evaporators, ovens, vacuum ovens, tray ovens, rotary ovens, and fluidized bed dryers. The isolated trans-1,4-naphthoquinone of formula (I), obtained in step (c) (i) may be dried for 6 hours, at a temperature range of 50-55° C.

In accordance with the present invention optional step (c) (ii) comprises recrystallization of the trans 1,4-naphthquinone of formula (I) from an organic solvent.

Suitable non limiting examples of organic solvents are: THF, acetone, acetonitrile, dioxane, ethanol, methanol, ethyl acetate, methyl acetate, and combination thereof. Preferably, the solvent used for crystallizing compound (I) is acetonitrile.

In a specific embodiment of the cis-1,4-napthaquinones of formula I, X is a chloride or hydroxyl and Y is a substituted or unsubstituted pyridine.

A particularly specific embodiment includes reacting cis-2-[4-(4-chlorophenyl)cyclohexyl]-3-hydroxy-1,4-naphthoquinone (1 equivalent) with sulfuric acid (30 equivalents), at 15-16° C., for less than one hour, pouring the mixture into ice and stirring for 20 minutes, collecting the solid by flirtation, washing with water, drying and recrystallizing from acetonitrile.

The invention further provides the epimerization of a mixture of cis and trans-compounds of formula (III) into the trans-compounds of formula (I), wherein X is as defined above.

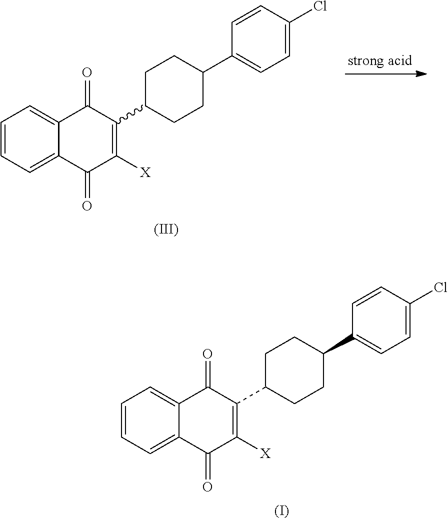

The process comprises:
a) reacting the compound of formula (III) with a strong acid,
b) quenching the reaction mixture; and
c) isolating the compound of formula (I).

Suitable non limiting examples of strong acids are: sulfuric acid and methanesulphonic acid, preferably sulfuric acid.

The molar ratio of the strong acid (e.g sulfuric acid) to the 1,4-naphthoquinone of formula (III) can be from about 4:1 to about 40:1, preferably about 30:1.

The process may be carried out at a temperature range of 5-35° C., preferably at 15-18° C. and the reaction mixture is stirred for less than one hour.

In accordance with the present invention quenching the reaction mixture, step (b), is carried out by purring the mixture into ice, and further stirring for 20 minutes. Step (b), may also be carried out by pouring the mixture into water at 0° C., and further stirring for 20 minutes.

In accordance with the present invention, after the reaction mixture is quenched, the isolation step (c) comprises the following steps:

(i) collecting the solid by flirtation, washing and drying to yield the 1,4-naphthoquinone of formula (I); and optionally

(ii) recrystallization of the obtained 1,4-naphthoquinone of formula (I).

The isolated 1,4-naphthoquinone of formula (I), obtained in step (c) (i) may be dried as described above.

In accordance with the present invention optional step (c) (ii) may comprises recrystallization of the trans 1,4-naphthquinone of formula (I) from an organic solvent.

Suitable non limiting examples of organic solvents for the recrystallization are: THF, acetone, acetonitrile, dioxane, ethanol, methanol, ethyl acetate, methyl acetate, and combination thereof. Preferably, the solvent used for crystallizing compound (I) is acetonitrile.

In a specific embodiment of the 1,4-napthaquinones of formula I, X is a chloride or hydroxyl and Y is a substituted or unsubstituted pyridine.

A particularly specific embodiment includes reacting a mixture of cis and trans-2-[4-(4-chlorophenyl)cyclohexyl]-3-hydroxy-1,4-naphthoquinone (1 equivalent) with sulfuric acid (30 equivalents), at 16-18° C., for less than one hour, pouring the mixture into ice and stirring for 20 minutes, collecting the solid by flirtation, washing with water and drying to obtain 2-[4-(4-chlorophenyl)cyclohexyl]-3-chloro-1,4-naphthoquinone.

The advantage of the novel process is the higher yield of the atovaquone compared to the processes described in the prior art and the easier isolation of the final product. When the cis isomer of atovaquone or its cis intermediates, are converted to the trans form by a simple method, using common reagents and mild reaction condition, it increases the efficiency of the process.

### Example 1

Cis-2-[4-(4-chlorophenyl)cyclohexyl]-3-hydroxy-1,4-naphthoquinone (1 g, 2.7 mmol) was stirred in 8 ml concentrated H₂SO₄ at 15-16° C. for 20 minutes. The reaction mixture was slowly poured into 30 g ice and further stirred for 20 minutes. The obtained solid was filtered and washed with water until the pH of the filtrate becomes in the range of 4 to 5. The resulting solid is dried at 50-55° C. for 6 hours to yield 0.85 g of trans-2-[4-(4-chlorophenyl)cyclohexyl]-3-hydroxy-1,4-naphthoquinone (85% yield, 95.5% purity).

### Example 2

The procedure of example 1 was repeated for cis-2-[4-(4-chlorophenyl)cyclohexyl]-3-chloro-1,4-naphthoquinone (1:0.007 ratio cis/trans, containing 50% of 4-(4-chlorophenyl)cyclohexyl-1-carboxylic acid) to obtain 2-[4-(4-chlorophenyl)cyclohexyl]-3-chloro-1,4-naphthoquinone (1:9.48 ratio cis/trans).

### Example 3

The procedure of example 1 was repeated for a mixture of cis and trans-2-[4-(4-chlorophenyl)cyclohexyl]-3-hydroxy-1,4-naphthoquinone (48:41.5 ratio cis/trans) to obtain 2-[4-(4-chlorophenyl)cyclohexyl]-3-hydroxy-1,4-naphthoquinone (95% yield, 4:85.5 ratio cis/trans).

### Example 4

The procedure of example 1 was repeated for a mixture of cis and trans-2-[4-(4-chlorophenyl)cyclohexyl]-3-(2-pyridine thiol)-1,4-naphthoquinone (1.9:1 ratio cis/trans) to obtain 2-[4-(4-chlorophenyl)cyclohexyl]-3-(2-pyridine thiol)-1,4-naphthoquinone (1:1.2 ratio cis/trans).

1. A process for the epimerization of cis-1,4-naphthoquinones of formula II, 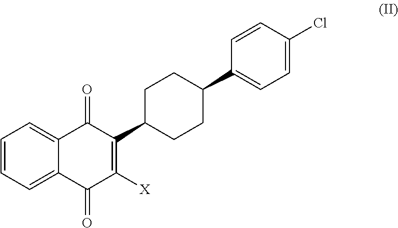
into the trans-1,4-naphthoquinones of formula I, 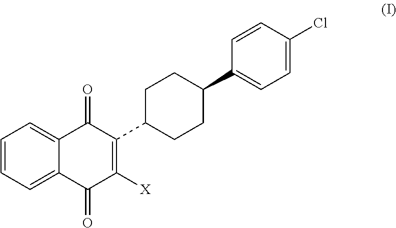
wherein X is a hydroxyl, a halogen or SY, and Y is substituted or unsubstituted aryl and heterocycloalkyl.
the process comprising:
a) reacting the compound of formula (II) with a strong acid,
b) quenching the reaction mixture; and
c) isolating the compound of formula (I).

2. The process of claim 1, wherein X is a hydroxyl or chloride and Y is a substituted or unsubstituted pyridine.

3. The process of claim 1, wherein said strong acid is selected from a group comprising of sulfuric acid and methanesulphonic acid.

4. The process of claim 3, wherein said strong acid is sulfuric acid.

5. The process of claim 1, wherein the molar ratio of the strong acid to the 1,4-naphthoquinone of formula (II) is from about 4:1 to about 40:1.

6. The process of claim 5, wherein the molar ratio of the strong acid to the 1,4-naphthoquinone of formula (II) is about 30:1.

7. The process of claim 1, wherein said reaction is conducted at a temperature range of 5° C. to 35° C.

8. The process of claim 7, wherein said reaction is conducted at 15-18° C.

9. The process of claim 1, wherein said reaction is quenched by pouring the mixture into ice or into water at 0° C.

10. The process of claim 1, wherein said isolation step comprises:
collecting the product by flirtation, washing and drying; and optionally recrystallizing the crude product.

11. The isolation step according to claim 10, wherein said product is further recrystallised from an organic solvent.

12. The isolation step according to claim 11, wherein the organic solvent is acetonitrile.

13. A process for the epimerization of a mixture of cis and trans-compounds of formula III, 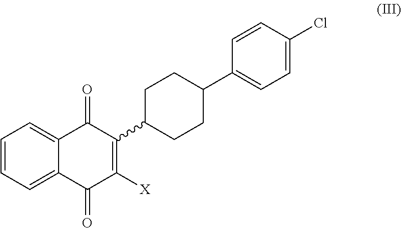
into the trans-compounds of formula I, 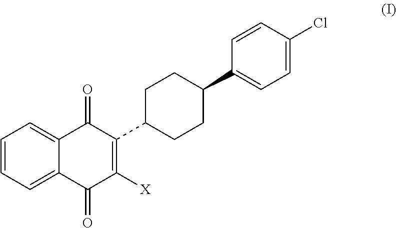
wherein X is a halogen, a hydroxyl and SY, and Y is substituted or unsubstituted aryl and heterocycloalkyl.
the process comprising:
a) reacting the compound of formula (II) with a strong acid,
b) quenching the reaction mixture; and
c) isolating the compound of formula (I).

14. The process of claim 13, wherein X is a hydroxyl or chloride and Y is a substituted or unsubstituted pyridine.

15. The process of claim 13, wherein said strong acid is selected from a group comprising of: sulfuric acid and methanesulphonic acid.

16. The process of claim 15, wherein said strong acid is sulfuric acid.

17. The process of claim 13, wherein the molar ratio of the strong acid to the compound of formula (III) is from about 4:1 to about 40:1.

18. The process of claim 17, wherein the molar ratio of the strong acid to the compound of formula (III) is about 30:1.

19. The process of claim 13, wherein said reaction is conducted at a temperature range of 5° C. to 35° C.,

20. The process of claim 19, wherein said reaction is conducted at 15-18° C.

21. The process of claim 13, wherein said reaction is quenched by pouring the mixture into ice or into water at 0° C.

22. The process of claim 13, wherein said isolation step comprises:
collecting the product by flirtation, washing and drying; and optionally recrystallizing the crude product.

23. The isolation step according to claim 22, wherein said product is further recrystallised from an organic solvent.

24. The isolation step according to claim 23, wherein the organic solvent is acetonitrile.
